(19) United States
(12) Patent Application Publication
(10) Pub. No.: US 2007/0049960 A1
(43) Pub. Date: Mar. 1, 2007

(54) Method of implanting a multi-lumen catheter

(75) Inventors: John Stephens, Perkiomenville, PA (US); J. Daniel Raulerson, Brewton, AL (US); Timothy Schweikert, Levittown, PA (US)

Correspondence Address: MONTE & MCGRAW, PC, 4092 SKIPPACK PIKE, P.O. BOX 650, SKIPPACK, PA, 19474, US

(73) Assignee: Medical Components, Inc., Harleysville, PA (US)

(21) Appl. No.: 11/525,763

(22) Filed: Sep. 22, 2006

Related U.S. Application Data
(62) Division of application No. 10/736,365, filed on Dec. 15, 2003.
(60) Provisional application No. 60/434,303, filed on Dec. 18, 2002.
(60) Provisional application No. 60/447,086, filed on Feb. 13, 2003.
(60) Provisional application No. 60/491,034, filed on Jul. 30, 2003.

(51) Int. Cl. A61B 17/00

(52) U.S. Cl. 606/190

(57) ABSTRACT

A catheter tunneling device (100) including a tunneler (110) including a proximal tip (112), a distal end (114), and an elongated body (118) extending between the proximal tip and the distal end. The device further includes an adapter (120) including a generally elongated body (122) having a distal end (126) and a proximal end (124) and a longitudinal axis (128) extending therethrough between the distal end and the proximal end. The proximal end (124) of the adapter (120) is connected to the distal end (114) of the tunneler (110). The distal end (126) of the adapter (120) comprises a connector for connecting a plurality of catheter lumens (200,202) thereto. A method of subcutaneously tunneling a catheter under a patient's skin using the tunneling device is also disclosed.

### CROSS REFERENCE TO RELATED APPLICATIONS

The present application claims priority from U.S. Provisional Patent Application Ser. No. 60/434,303, filed Dec. 18, 2002; from U.S. Provisional Patent Application Ser. No. 60/447,086, filed Feb. 13, 2003; and from U.S. Provisional Patent Application Ser. No. 60/491,034, filed Jul. 30, 2003.

### FIELD OF THE INVENTION

The present invention relates to a tunneler device for subcutaneously tunneling a plurality of catheter lumens under a patient's skin after a portion of the lumens have already been inserted into the patient's blood vessel.

### BACKGROUND OF THE INVENTION

Catheters for the introduction or removal of fluids may be located in various venous locations and cavities throughout the body of a patient for introduction of fluids to the body or removal of fluids from the body. Such catheterization may be performed by using a single catheter having multiple lumens. A typical example of a multiple lumen catheter is a dual lumen catheter in which one lumen introduces fluid and the other lumen removes fluid. An example of such a multiple catheter is the SPLIT-CATH® catheter.

Generally, to insert any catheter into a blood vessel, the vessel is identified by aspiration with a long hollow needle in accordance with the well known Seldinger technique. When blood enters a syringe attached to the needle, indicating that the vessel has been found, a thin guide wire is then introduced, typically through a syringe needle or other introducer device into the interior of the vessel. The introducer device is then removed, leaving the end portion of the guide wire that has been inserted into the vessel within the vessel and the opposing end of the guide wire projecting beyond the surface of the skin of the patient. At this point, several options are available to a physician for catheter placement. The simplest is to pass a catheter into the vessel directly over the guide wire. The guide wire is then removed, leaving the catheter in position within the vessel. However, this technique is only possible in cases where the catheter is of a relatively small diameter, made of a stiff material, and not significantly larger than the guide wire. For example, this technique may be used to insert small diameter dual lumen catheters into a patient. If the catheter to be inserted is significantly larger than the guide wire, a dilator device is passed over the guide wire to enlarge the hole. The dilator device is then removed, and the catheter is then passed over the guide wire into the vessel. The guide wire is then removed.

For chronic catheterization, in which the catheter is intended to remain inside the patient for an extended period of time, such as for weeks or even months, it is typically desired to subcutaneously tunnel the catheter using various tunneling techniques. The catheter is typically tunneled into the patient prior to inserting the catheter into the patient's vein. However, depending on the patient or the implanting surgeon's skill, there may be times when it is more advantageous to perform the tunneling after the catheter is implanted in the patient. For some catheters, though, such as multiple lumen catheters with a hub and with bonded luers on the proximal ends of the catheters, it is impractical to perform the tunneling after the catheter is installed in the patient.

It would be beneficial to provide a catheter assembly and insertion tools that provide a surgeon with alternative installation procedures for installing the catheter that better suit either the patient's needs or the surgeon's skills. Such an alternative catheter assembly is the multi-lumen catheter disclosed in U.S. patent application Ser. No. 10/695,178, filed on Oct. 28, 2003.

In order to be able to perform the tunneling after the distal end of the catheter assembly is inserted into the patient, the proximal ends of each catheter must be attached to a tunneler device adapted to pull the proximal end of each catheter through the tunnel. After tunneling, the proximal ends of the catheters must be disconnected from the tunneler device connected to a catheter hub. It would be beneficial to provide a tunneler device that allows for the simultaneous tunneling of the proximal ends of each catheter in a multi-lumen catheter assembly.

### BRIEF SUMMARY OF THE INVENTION

Briefly, the present invention provides a catheter tunneling adapter. The adapter is comprised of a generally elongated body having a distal end and a proximal end and a longitudinal axis extending therethrough between the distal end and the proximal end. The proximal end includes a connection means for connecting a catheter tunneler thereto. The distal end comprises a connection means for connecting a plurality of catheter lumens thereto.

Also, the present invention provides a catheter tunneling device. The device is comprised of a proximal portion including a proximal tip, a distal end, and an elongated body extending between the proximal tip of the proximal portion of the catheter tunneling device and the distal end of the proximal portion of the catheter tunneling device. The device further includes a distal portion including a generally elongated body having a distal end and a proximal end and a longitudinal axis extending therethrough between the distal end of the distal portion of the catheter tunneling device and the proximal end of the distal portion of the catheter tunneling device. The proximal end of the distal portion is connected to the distal end of the proximal portion. The distal end of the distal portion comprises a connection means for connecting a plurality of catheter lumens thereto.

Further, the present invention provides a method of inserting a catheter having a plurality of lumens into a patient. The method is comprised of inserting a distal end of each of the plurality of lumens into a blood vessel in the patient; connecting a proximal end of each of the plurality of lumens to a tunneling device; forming a subcutaneous tunnel with the tunneling device; drawing the proximal ends of each of the plurality of lumens simultaneously through the tunnel; disconnecting the tunneling device from the proximal ends of each of the plurality of lumens; and connecting the proximal ends of each of the plurality of lumens to a catheter hub.

### BRIEF DESCRIPTION OF THE DRAWINGS

The accompanying drawings, which are incorporated herein and constitute part of this specification, illustrate the presently preferred embodiment of the invention, and, together with the general description given above and the detailed description given below, serve to explain the features of the invention. In the drawings:

FIG. 1 is a side view of a catheter tunneler assembly, including a catheter tunneler and a catheter tunneler adapter, according to a preferred embodiment of the present invention.

FIG. 2 is a perspective view of the catheter tunneler adapter of FIG. 1.

FIG. 3 is a side view of the catheter tunneler adapter with catheters of FIG. 1.

FIG. 4 is a sectional view of the catheter tunneler adapter with catheters taken along line 4-4 of FIG. 3.

FIG. 5 is a top plan view of a catheter tunneler adapter according to an alternate embodiment of the present invention.

FIG. 6 is a top plan view of the catheter tunneler adapter shown in FIG. 5, showing interior passageways.

FIG. 7 is a top view of an alternate embodiment of a catheter tunneler adapter, with catheters attached.

FIG. 8 is a flow chart illustrating the steps of inserting a catheter assembly using the catheter tunneler assembly according to the present invention.

FIG. 9 is a side view of the catheter tunneler adapter with catheters being primed by a syringe.

### DETAILED DESCRIPTION OF THE PRESENT INVENTION

In the drawings, like numerals indicate like elements throughout. Certain terminology is used herein for convenience only and is not to be taken as a limitation on the present invention. The words “proximal” and “distal” refer to directions away from and closer to, respectively, the insertion tip of a catheter adapted to connect to the tunneler device according to the present invention. The terminology includes the words above specifically mentioned, derivatives thereof, and words of similar import. The following describes a preferred embodiment of the invention. However, it should be understood based on this disclosure, that the invention is not limited by the preferred embodiment described herein.

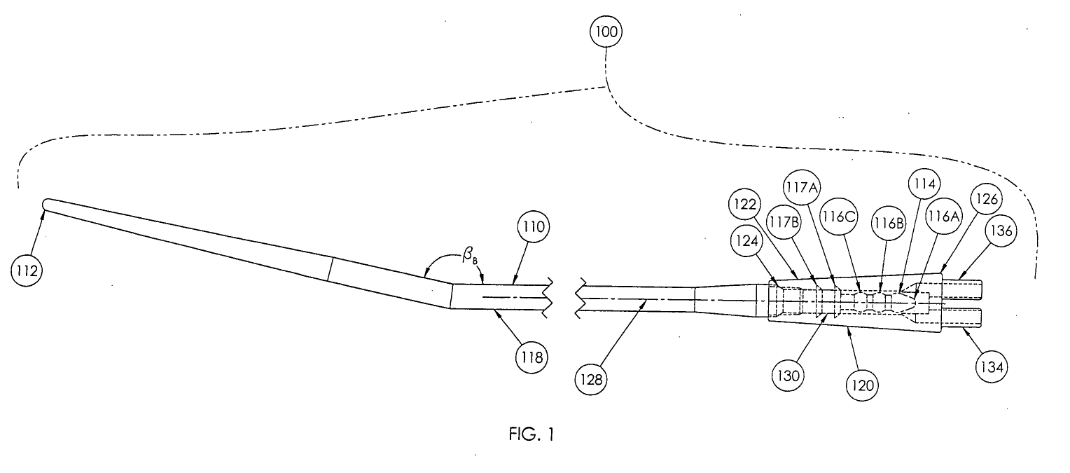

Referring to FIG. 1, a catheter tunneling device 100 according to a preferred embodiment of the present invention is shown. The tunneling device 100 includes a proximal portion, or tunneler 110, which is comprised of a proximal tip 112 and a distal end 114. Preferably, the distal end 114 includes at least one, and more preferably, a plurality of bulbous projections 116a, 116b, 116c. Preferably, the bulbous projection 116a is generally conically shaped. While three bulbous projections 116a, 116b, 116c are shown in FIG. 1, those skilled in the art will recognize that more or less than three bulbous projections 116a, 116b, 116c may be used. A plurality of barbed projections 117a, 117b are disposed along the tunneler 110 proximally of the bulbous projections 116a, 116b, 116c.

The tunneler 110 has an elongated body 118 that extends between the proximal tip 112 and the distal end 114. The elongated body 118 preferably bends at a predetermined location along the length of the body 118. As shown in FIG. 1, the body 118 is bent at an angle β_{B} of approximately 12 degrees; although those skilled in the art will recognize that the body 118 may also be bent more or less than 12 degrees. Preferably, the tunneler 110 is of unitary construction and is preferably constructed from stainless steel, although those skilled in the art will recognize that the tunneler 110 may be constructed from other suitable materials.

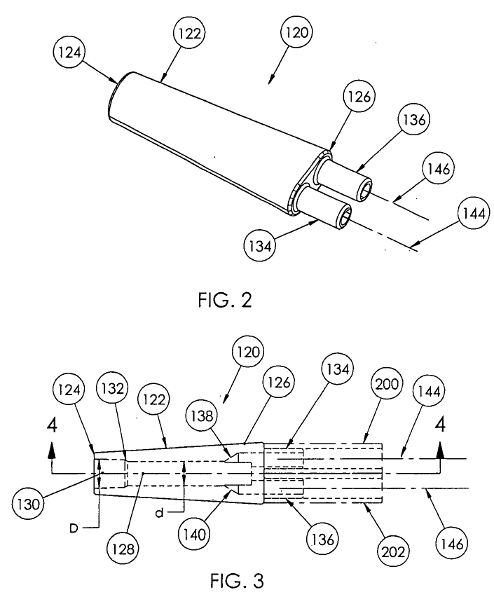

Referring now to FIGS. 1-4, the tunneling device 100 further includes a distal portion, or adapter 120. The adapter 120 is comprised of a generally elongated tubular body 122 having a generally circular proximal end 124, which tapers to a generally oblong distal end 126. A longitudinal axis 128 extends between the proximal end 124 and the distal end 126. Preferably, the proximal and distal ends 124, 126 of the adapter 120 are tapered to help ensure smooth tunneling, in both antegrade and retrograde tunneling procedures, as may be preferred by the inserting physician. The proximal end 124 of the adapter 120 smoothly transitions the distal end of the tunneler 110 to the adapter 120 to prevent skin subcutaneous tissue from being snagged and torn during tunneling.

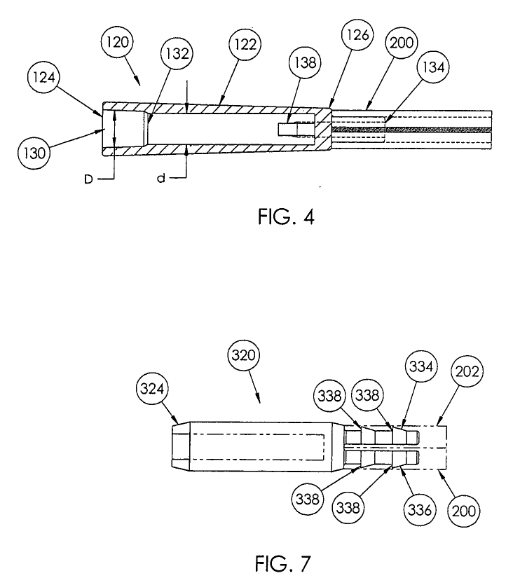

Referring now to FIGS. 3 and 4, the proximal end 124 of the adapter 120 includes a connection means for connecting the distal end 114 of the tunneler 110 thereto. The connection means comprises a generally tubular passageway 130 extending generally longitudinally through the tubular body 122 from the proximal end 124 toward the distal end 126 of the adapter 120. The passageway 130 is sized to accept and securely retain the distal end 114 of the tunneler 110. Preferably, the passageway 130 has a mininum first diameter “D” at the proximal end 124 and a second diameter “d”, smaller than the diameter “D”, distal of the proximal end 124. The passageway 130 tapers along a taper 132 between the first diameter D and the second diameter “d”.

The distal end 126 of the adapter 120 includes a connection means for connecting a plurality of catheter lumens 200, 202 thereto. The connection means comprises a like plurality of extensions 134, 136, which extend distally from the distal portion 126. Referring still to FIGS. 3 and 4, each of the plurality of extensions 134, 136 is sized and shaped to retain one of the catheter lumens 200, 202 thereon. While two extensions 134, 136 and two catheter lumens 200, 202 are shown, those skilled in the art will recognize that a plurality of more than two extensions 134, 136 and a like plurality of more than two catheter lumens 200, 202 may be used.

The extensions 134, 136 are disposed on opposing sides of the longitudinal axis 128. Each of the plurality of extensions 134, 136 comprises a longitudinal axis 144, 146, respectively. The extensions 134, 136 preferably have generally circular cross-sections. The cross-sections are shaped to accept generally circular lumens 200, 202, such as the lumens in the SC-4™ catheter, manufactured by Medical Components of Harleysville, Pa. However, those skilled in the art will recognize that the catheter lumens 200, 202 may also be generally D-shaped in cross-section. Consequently, the corresponding extensions 134, 136 may also be generally D-shaped in cross-section to accept the D-shaped lumens. Likewise, those skilled in the art will recognize that the plurality of extensions 134, 136 could comprise a cross-section of any shape that would match the like catheter lumens 200, 202 that were to be attached thereto.

The plurality of extensions 134, 136 may extend longitudinally away from the distal end of the adapter body 122. In such a configuration, each of the plurality of extensions 134, 136 is generally parallel to the other. In addition, the longitudinal axes 144, 146 of the plurality of extensions 134, 136 may be generally parallel to the longitudinal axis of the adapter 128, or alternatively, there may exist an angle between the longitudinal axis of the adapter 128 and the plurality of extensions 134, 136.

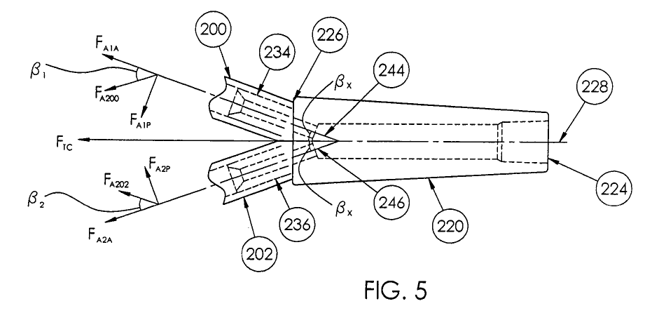

However, in an alternate embodiment of an adapter 220, shown in FIG. 5, there exists an angle β_{X} between the longitudinal axis 244, 246 of each extension in the plurality of extensions 234, 236 and the longitudinal axis 228 of the adapter 220. The angle β_{X} may be between approximately 0° and approximately 89°. In an embodiment wherein the plurality of extensions 234, 236 are connected to the first and second lumens 200, 202, respectively, an angle β_{X} between approximately 17° and approximately 23° is preferred, but not required. The catheter lumens 200, 202 on the extensions are shown in phantom in FIG. 5. Preferably, the material from which the extensions 234, 236 are constructed allows the extensions 234, 236 to bend toward each other during tunneling in order to minimize dilation of the tunnel and exit site.

As a result of the angle β_{X} between the longitudinal axis 244, 246 of each of the plurality of extensions 234, 236 and the longitudinal axis 228 of the adapter 220, a tensile force FTC exerted on the catheter lumens 200, 202, which would tend to pull each catheter lumen 200, 202 distally away from the adapter 220, is mitigated. The tensile force FTC on the catheter lumens 200, 202, generated by friction between the catheter lumens 200, 202 and the flesh of the patient during tunneling, are generally along the same or a similar line as the longitudinal axis 228 of the adapter 220. Therefore, having extensions 234, 236 with longitudinal axes 244, 246 parallel to the longitudinal axis 228 of the adapter 220 may allow the tensile forces FTC on the catheter lumens 200, 202 to pull the lumens 200, 202 axially along the extensions 234, 236, away from the catheter tunneling adapter 220 in the distal direction. By forming an angle β_{X} between at least one of each of the extensions 234, 236 and the longitudinal axis 228 of the adapter 220, angles β₁, β₂ between the tensile force FTC on the catheter lumens 200, 202 and one or both of the extensions 234, 236 respectively are established. The tensile force FTC on the catheter lumens 200, 202 is distributed between each of the lumens 200, 202 extending therefrom. The result of the angle β₁, β₂ between the tensile force FTC on the catheter lumens 200, 202 and the extensions 234, 236 reduces the force pulling the plurality of lumens 200, 202 axially along the plurality of extensions 234, 236 to only the cosine of the angle β₁, β₂, multiplied by the magnitude of the axial force on the lumen F_{A200}, F_{A202}. The sine component of the axial force F_{A200}, F_{A202} is the perpendicular force F_{A1P}, F_{A2P}. Therefore, by distributing the axial force F_{A200}, F_{A202} on each individual lumen 200, 202 into axial F_{A1A}, F_{A2A} and perpendicular force F_{A1P}, F_{A2P} components, the force pulling the lumens 200, 202 directly off of the extensions 234, 236 is reduced, thereby reducing the likelihood of the lumen 200, 202 being pulled from the extension 234, 236 during tunneling.

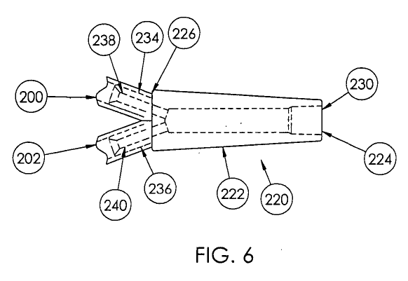

Referring now to FIG. 6, the extensions 234, 236 are inserted into and bonded directly to each respective lumen 200, 202. The extensions 234, 236 each include a passageway 238, 240 respectively, that fluidly connects the distal end of the passage 230 with each of the catheter lumens 200, 202 when the catheter lumens 200, 202 are disposed over the extensions 234, 236, respectively.

In an alternate embodiment of an adapter 320 shown in FIG. 7, extensions 334, 336 includes at least one, and preferably at least two barbs 338 that generally circumscribe each extension 334, 336. The barbs 338 extend generally toward the proximal end 324 of the adapter 320. The barbs 338 extend in this direction to more securely retain the catheter lumens 200, 202 when the catheter lumens 200, 202 are disposed over the extensions 334, 336. In this embodiment, the lumens 200, 202 preferably have “D-shaped” cross-section, although those skilled in the art will recognize that circular or other cross-sectional shapes may be used.

Preferably, the adapter 120, 220, 320 is of unitary construction and is constructed from a polymer, such as polypropylene or polyurethane, although those skilled in the art will recognize that the adapter 120, 220, 320 may be of composite construction and may be constructed from other suitable materials as well.

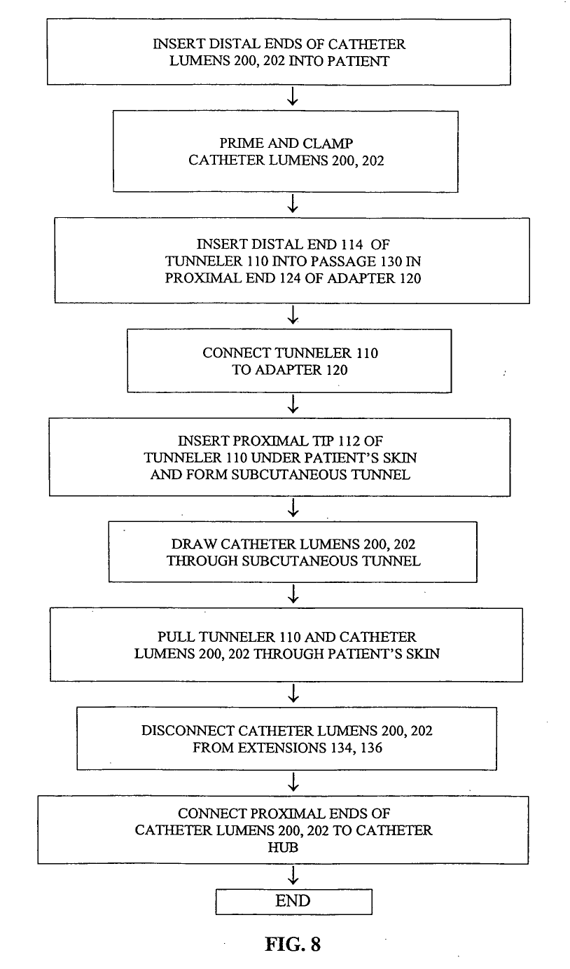

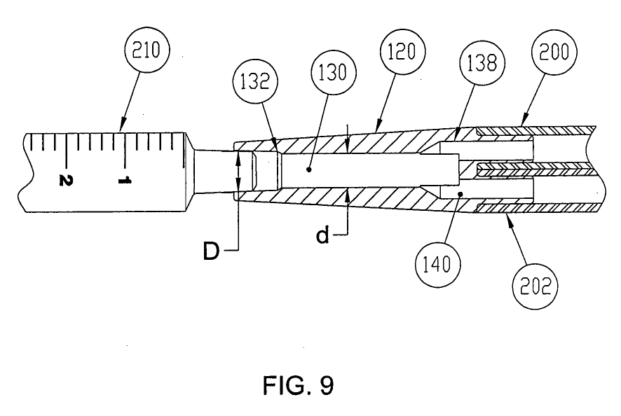

Referring to the flow chart of FIG. 8, to use the device 100, distal ends (not shown) of the catheter lumens 200, 202 are surgically inserted into a patient's blood vessel according to known techniques. The proximal ends of the catheter lumens 200, 202, are fixedly connected to the adapter 120, such as by solvent bonding or other suitable bonding method. Preferably, the catheter lumens 200, 202 are primed prior to insertion, such as with a saline solution or other suitable priming fluid. Referring now to FIG. 9, a syringe 210 is inserted into the proximal end of the passage 130. The taper 132 of the passageway 130 at the proximal end seals the syringe 210 in the passageway 130 along the first diameter D so that the priming solution does not readily leak from the boundary between the syringe 210 and the passageway 130. The taper 132 also acts as a stop to prevent further insertion of the syringe 210 into the passageway 130. Preferably, the second lumen 202 is clamped, such as with a known clamping device, and the solution is dispensed from the syringe 210 into the passageway 130, through the passage 138, and into the first catheter lumen 200. The first catheter lumen 200 is then clamped and the second catheter lumen 202 is unclamped. The priming solution is then dispensed from the syringe 210 into the passageway 130, through the passage 140, and into the second catheter lumen 202. Prior to removing the syringe 210, the first catheter lumen 200 is clamped to prevent the priming solution from leaking out the proximal end 124 of the adapter 120 after the syringe 210 is removed.

Referring back to the flow chart of FIG. 8, the syringe 210 is removed and the catheter lumens 200, 202 are inserted into the patient according to known techniques. The distal end 114 of the tunneler 110 is next inserted into the passageway 130 in the proximal end 124 of the adapter 120. The conical shape of the bulbous projection 116a aids in inserting the distal end 114 of the tunneler 110 into the passageway 130. The bulbous projections 116a, 116b, 116c easily slide through the passageway 130 along the first diameter “D”, but engage the side of the passageway 130 along the second diameter “d” in an interference fit. Due to the material from which the adapter 120 is constructed, the side of the passageway 130 deforms to allow the bulbous projections 116 to be inserted into the second diameter “d”. The barbed projections 117a, 117b dig in to the sides of the passageway 130, restricting the ability of the distal end 114 of the tunneler 110 to be removed from the passageway 130.

The proximal tip 112 of the tunneler 110 is inserted into the patient's skin. The entire tunneling device 100 and the proximal ends of the catheter lumens 200, 202 are subcutaneously drawn under a length of the patient's skin. The proximal tip 112 of the tunneler 110 is directed toward the surface of the patient's skin and the entire tunneling device 100 and the proximal ends of the catheter lumens 200, 202 are pulled out of the patient's skin. The catheter lumens 200, 202 are separated from the tunneling device 100 by cutting the tunneling device 100 from the catheter lumens 200, 202, such as along a cut line (not shown) marked on each respective catheter lumen 200, 202.

The catheter lumens 200, 202 are next connected to a catheter hub (not shown), such as the catheter hub disclosed in U.S. patent application Ser. No. 10/691,331 filed on Oct. 22, 2003 which is owned by the Assignee of the present invention.

Those skilled in the art will recognize that the adapters 220, 320 may be used in a similar manner to the method described above with respect to the adapter 120.

While a preferred order of steps of subcutaneously tunneling the catheter lumens 200, 202 is described above, those skilled in the art will recognize that the order of steps of tunneling can be rearranged, such as for example, by first connecting the tunneler 110 to the adapter 120 and then by connecting the lumens 200, 202 to the respective extensions 134, 136 prior to tunneling. Alternatively, the adapter 120 may be first connected to the proximal ends of the catheter lumens 200, 202, prior to inserting distal ends of the catheter lumens 200, 202 into the patient's blood vessel.

Alternatively, a retrograde tunnel may be formed by inserting the proximal tip 112 of the tunneler 110 under the patient's skin proximate to the chest cavity and tunneling upward toward the catheter incision site. While the proximal tip 112 of the tunneler 110 is sufficiently sharp to tunnel through the patient's skin tissue, the proximal tip 112 of the tunneler 110 is not sharp enough to puncture the catheter lumens 200, 202 in the event that the inserting physician accidentally hits the catheter lumens 200, 202 during tunneling. After the tunnel is formed, the adapter 120 is connected to the tunneler 110 as described above, and the tunneler 110 and adapter 120, along with proximal ends of the catheter lumens 200, 202, are pulled through the tunnel.

Further, while the tunneling device 100 is described herein as two separate parts, namely a tunneler 110 and an adapter 120, those skilled in the art will recognize that the tunneling device 100 may be a single part.

It will be appreciated by those skilled in the art that changes could be made to the embodiment described above without departing from the broad inventive concept thereof. It is understood, therefore, that this invention is not limited to the particular embodiment disclosed, but it is intended to cover modifications within the spirit and scope of the present invention as defined by the appended claims.

What is claimed is:

1 to 32. (canceled)

33. A method of implanting a catheter having a plurality of lumens into a patient comprising:
inserting a distal end of each of the plurality of lumens into a blood vessel in the patient;
connecting a proximal end of each of the plurality of lumens to a tunneling device;
forming a subcutaneous tunnel with the tunneling device;
drawing the proximal ends of each of the plurality of lumens simultaneously through the tunnel;
disconnecting the tunneling device from the proximal ends of each of the plurality of lumens; and
connecting the proximal ends of each of the plurality of lumens to a catheter hub.

34. The method according to claim 33, wherein connecting a proximal end of each of the plurality of lumens comprises connecting a proximal end of each of a first and a second lumen.

35. The method according to claim 33, wherein connecting a proximal end of each of the plurality of lumens to a tunneling device comprises connecting a distal end of a tunneling adapter to the proximal end of each of the plurality of lumens and then connecting a proximal end of the tunneling device adapter to the distal end of a tunneler.

36. The method according to claim 35, further comprising, after connecting the distal end of the tunneling adapter to the proximal end of each of the plurality of lumens, injecting a priming solution into the catheter lumens through the distal end of the tunneling adapter.
